(19) United States
(12) Patent Application Publication
(10) Pub. No.: US 2014/0188287 A1
(43) Pub. Date: Jul. 3, 2014

(54) iComfort: Method to measure and control your micro-climate using a smart phone

(71) Applicant: Ashok Sabata, (US)

(72) Inventor: Ashok Sabata, (US)

(21) Appl. No.: 13/732,160

(22) Filed: Dec. 31, 2012

(51) Int. Cl. F24F 11/00; G01N 33/00

(52) U.S. Cl. CPC F24F 11/0086; G01N 33/0004
USPC 700/276; 73/29.02

(57) ABSTRACT

Temperature, relative humidity and air quality define comfort indoors. Individuals judge comfort by how the environment feels and smells. Heating, ventilation and air conditioning (HVAC) equipments are used to control the indoor climate and thereby control comfort. Most homes and offices are normally equipped with just a sensor to show the temperature level. It is rare to find humidity sensors and temperature sensors in every room in homes and offices so controlling the environment locally is not possible. Air quality, another parameter that influences comfort, is measured by the presence of volatile organic compounds or particles that can now be identified using more sophisticated monitors. An invention is described here that will make it easy and affordable to measure and control the temperature, relative humidity and air quality using a smart phone, that is commonplace now, thereby controlling the comfort level.

### BACKGROUND OF THE INVENTION

In the U.S. more then 50% of home energy usage is related to heating and cooling. Comfort and energy efficiency can sometimes be in conflict but with advances in technologies optimal ways are now available to reduce energy consumption while improving comfort. Comfort and energy efficiency can be achieved by better control and regulation of heating, ventilation, and air conditioning (HVAC) equipment. Thermostats are used to control the HVAC equipment. There are thermostats that offer programming abilities for balancing user comfort and energy savings. However, such ability oftentimes are difficult to use therefore the users will frequently resort to default programs.

In recent years several new thermostats have been launched that attempt to make the thermostat user interface easier to use. However, all of these still use the same paradigm of HVAC—controlled via thermostat with a programmable interface with a display. The paradigm is still the same even with wireless and remote monitoring capability, that is, have the HVAC controlled by a wall mounted thermostat with display.

### BRIEF SUMMARY OF THE INVENTION

The current invention as compared to prior art replaces thermostats with or without display with a central communication unit (CCU) thereby eliminating the modern wall mounted thermostats with state-of-the-art user interfaces.

An embodiment of the present invention provides a method for monitoring the temperature, humidity and air quality of indoor air to measure comfort level. The monitoring is accomplished using single or multiple low power wireless sensors, placed indoors in one or many rooms. The system also includes a device (that will replace a thermostat or attach to an existing thermostat) for control of the HVAC. The device has a wireless module for communications, memory to store settings and controls for the HVAC. The user can interact with the device using a smart phone or a computer in the wireless mode.

In one embodiment the CCU uses WiFi wireless that is based on 802.11. The communication unit may include a memory module that stores sensor set points that are set from the handheld smart device or from a computer; and a plurality of heating, ventilation, and air conditioning (HVAC) wire connectors coupled to the communication unit, the communication unit being configured to send at least one control signal through the HVAC wire connectors to an HVAC system based at least in part on a comparison of the measured ambient temperature and the setpoint temperature value.

In some embodiments, a method for control of an HVAC system by a communication unit is provided. The communication unit may include: a memory module that stores sensor set points that are set from the handheld device or from a computer, a processing system to control the HVAC, and a plurality of HVAC wire connectors. The method may include: measuring ambient conditions using a myriad of sensors such as temperature, relative humidity and other air quality related sensors. The sensors are wireless sensors that transmit the sensor values to the communication unit. The CCU use the setpoints that are on the onboard memory of the CCU to send at least one control signal through the HVAC wire connectors to the HVAC system based at least in part on a comparison of the measured ambient sensor values with the setpoint values.

The present invention represents a substantial advance over prior systems and methods for monitoring indoor environment and controlling HVAC. In one embodiment the environmental monitoring includes temperature, relative humidity and air quality. The air quality is monitored using CO2, air particles and any other harmful air borne entity. All the sensors used for monitoring are wireless sensors that are placed strategically indoors to get the best comfort while using the HVAC most efficiently. The CCU controlling the HVAC communicates with the sensors as well as the handheld device. The wireless network of the communication device, sensors and handheld device is used define the setpoints to control the HVAC. Specifically the handheld smart device (such as a smart phone) is used to input temperature setpoints to control the heating and air conditioning; to input relative humidity setpoints to control ventilation; to input air quality setpoints to control ventilation. The setpoints are suggested dynamically by using an algorithm on the smart handheld device that uses past historical behavior and known relationship between comfort, energy efficiency and HVAC settings. These advantages, and other advantages and benefits of the present invention, will become apparent from the Detailed Description of the Invention herein below.

### BRIEF DESCRIPTION OF THE DRAWINGS

For the present invention to be clearly understood and readily practiced, the present invention will be described in conjunction with the following figures, wherein:

FIG. 1 is a diagram illustrating the elements of the monitoring and control system;

FIG. 2 is a diagram illustrating the components of the central communication unit (CCU);

FIG. 3 is a diagram of the wireless sensor package used indoors; and

FIG. 4 is a schematic of how to use the smart phone for monitoring and generating setpoints to control HVAC;

### DETAILED DESCRIPTION OF THE INVENTION

While the present invention will be described more fully it is to be understood at the outset of the description which follows that persons of skill in the appropriate arts may modify the invention herein described while still achieving the favorable results of this invention. Accordingly, the description which follows is to be understood as being a broad, teaching disclosure directed to persons of skill in the appropriate arts, and not as limiting upon the present invention.

First briefly in overview, the present invention according to one or more embodiments are systems, methods, computer program products, and related business methods for the indoor environment comfortable by controlling one or more HVAC systems using a handheld smart device based on one or more wireless sensing units (WSU), each WSU unit transmitting sensor data to a central communication unit (CCU) with no display, that uses the sensor data in combination with the stored setpoints in its memory to control the HVAC, while recording the total duration the HVAC runs, the total time the HVAC running is used for optimizing energy usage. The term CCU replaces the traditional “thermostat” as the CCU controls the HVAC not just using temperature but also using relative humidity, air quality and other energy and comfort related parameters. The CCU is powered with the current drawn from the control wires using a charging circuit which provides nominal voltage of 3.6 Volts. The sensor data is always stored on the WSU memory, until it is wirelessly transmitted periodically to the handheld smart device where the data is mined for trends and dynamically creating setpoints. For multiple persons to be able to control the CCU with multiple smart devices all the historical sensor readings, the dynamically determined setpoint information as well as the setpoint information is put in a remote server that synchronizes with all the smart devices recognized by the CCU. Another goal of the current invention is to optimize the energy efficiency by deriving usage patterns, recommending setpoints and providing user to locally as well as remotely control the HVAC.

FIG. 1 illustrates the components of the system that includes the central communication unit that communicates using WiFi acting as an access point, sensors that can be wall powered or battery powered, smart phone with an application for storing setpoints on the CCU. The sensors include temperature, relative humidity, air quality measured based on CO2 level and other health and comfort related sensors.

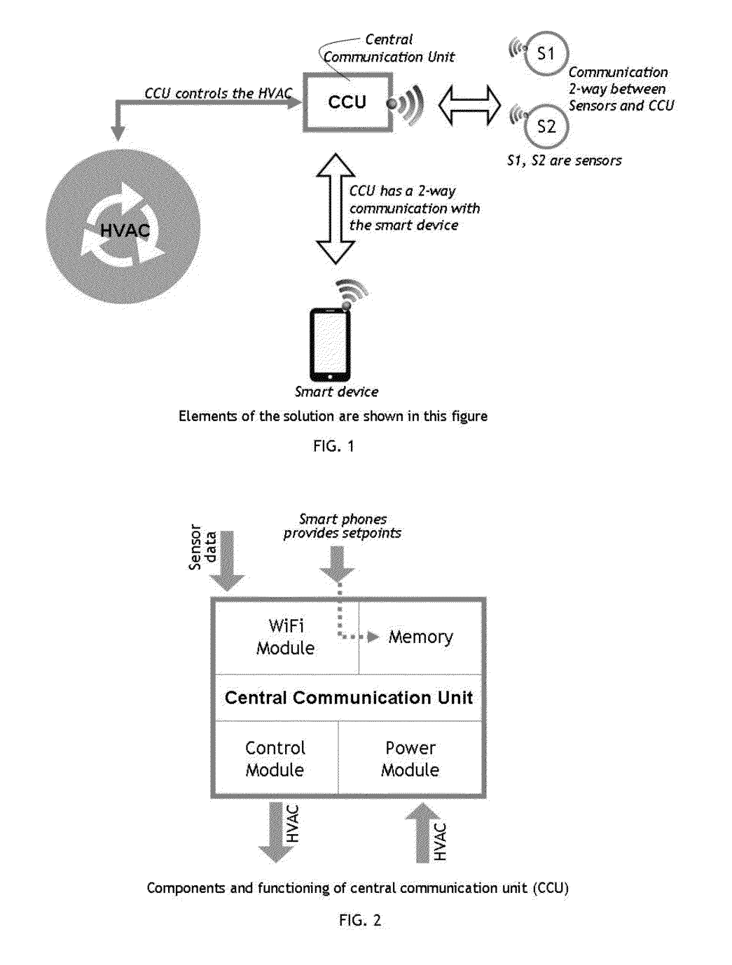

The current invention proposes using a communication unit with no wall mounted display or user interface to control the HVAC. FIG. 2 schematically shows the components of the CCU. The communication module uses WiFi to communicate with one or many sensors wherein the sensors act as clients and connect to the CCU to transmit the sensor readings to be used by the Control unit in the CCU to compare with the setpoints in the memory for control of the HVAC. There are 3 controls on the HVAC unit—turn ON/OFF heat, turn ON/OFF the air condition and turn ON/OFF the ventilation. Temperature sensors are used to create setpoints for the heating and AC. The ventilation is controlled based on setpoints for RH and air quality. The smart device also acts as a client to connect to the CCU to set the setpoints as well as connect the sensors through the CCU for viewing of live sensor data. The CCU could also connect to the internet and pass through information, such as sensor readings and the set points to a server for the purpose of remote monitoring. The CCU is powered directly by scavenging power from the control wires to the HVAC unit using the power module that gives a nominal voltage output of 3.6V, provides up to 200 mA current for wireless reception and transmission and powers the control unit controlling the HVAC.

The components of the sensor unit includes a WiFi module for communication, the sensors such as temperature, relative humidity and air quality (such as CO2 level) and power that can either be from a 3.6V Li battery or directly wall power using an adaptor. Battery power is used when the sensor is a mobile sensor that can be moved around and especially useful when improving the comfort of persons in the room. The sensor data can be used to view live data and change setpoints or store in a database for further analysis to recommend optimum setpoints taking comfort and energy efficiency into consideration. FIG. 3 depicts the two possible sensor data flow methods to either view sensor data or store for analysis. The sensor data can flow through the CCU to the smart device for live local sensor read out. Alternatively, the sensor data can also pass from the sensor through the CCU to a destination server on the internet where it is stored. Other sensors can be easily added to the known sensors such as T and RH for indoor use; in particular CO2 sensors and new upcoming sensors that measure air particles, allergens and other air borne entities that have effect on human health.

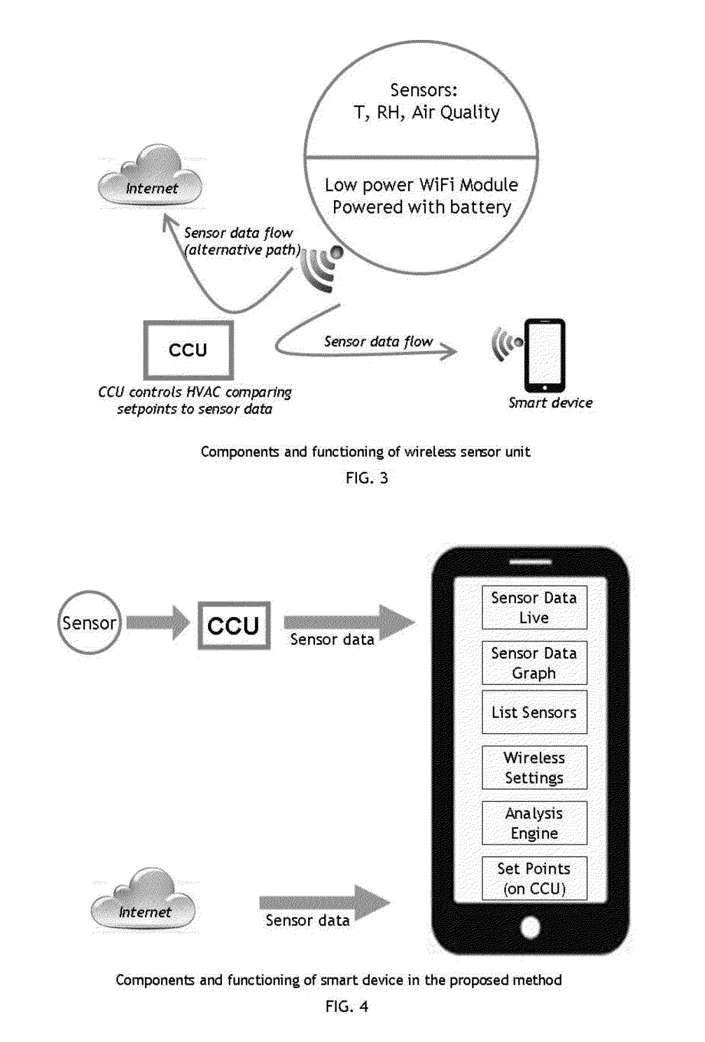

The current invention eliminates the need for a display or a user interface used in exiting thermostats thereby reducing cost while moving all monitoring, analysis and configuration activity to the smart handheld device such as a smart phone that is nowadays ubiquitous. Many smart phones have considerable processing power (more then any wall mounted thermostats) that gives considerable computing power to the user for algorithms that can help in optimizing comfort versus energy efficiency. FIG. 4 illustrates the dataflow to the smart device which is then processed to provide setpoints to the CCU for control of the HVAC. When the user is indoors and within the WiFi range (less then 100 ft) of the CCU, the user can connect to the CCU and view the live sensor data with the data path going from the sensor thru the CCU to the smart device. If the user selects the ability to monitor remotely the sensor data will travel from the sensor thru the CCU to the internet to a destination server for storage in a database. The smart device can then be used to view the sensor data from anywhere. If the user selects local monitoring only and the user is not within range the sensor data remains onboard the sensor device until the smart device is available for data upload from the sensor.

The current invention addresses comfort as a function of temperature, relative humidity and air quality. The smart device computes comfort from historical sensor data, and known information on comfort that are based on the interplay of T/RH and dynamic sensor readings, to determine the setpoints. The smart device then transmits the setpoints to CCU which then stores it in the memory for control of the HVAC. The preferred smart device is one that is now commonplace such as the iPhone/iPod touch or an android based system. It is envisioned that the ability to view the sensor readings and the HVAC functioning (e.g. how often it turns on/off) will bring an awareness to the user that can be leveraged to improve comfort and energy efficiency.

What is claimed is:

1. A method for monitoring of temperature, relative humidity and air quality of indoor air to measure the comfort level, the method comprising: sensors that measure temperature, relative humidity and air quality; using wireless technology that will work with the wireless networks used in homes and offices; transmitting the sensor measurement to a smart device; displaying the information as a graph showing changes in the environment over time.

2. The method of claim 1 wherein the wireless technology used is 802.11 b/g/n commonly used in homes and offices.

3. The method of claim 1 wherein the wireless sensors are battery powered with a AA Lithium battery life or a rechargeable LiPo battery life of more then 1 year.

4. The method of claim 1 wherein the wireless sensors communicate and are controlled with the handheld smart device via a central communication unit that replaces the traditional thermostats

5. The method of claim 1 wherein the wireless sensors communicate and are controlled with the handheld smart device remotely via the internet.

6. The method of claim 1 wherein analyzing sensor data is accomplished on the handheld smart device.

7. A method for controlling the temperature, relative humidity and air quality of indoor air to improve the comfort level, the method comprising: a wireless central communication unit (CCU) that controls the heating, ventilation and air conditioning equipment or system; a smart device connecting to CCU directly or remotely, and putting the setpoints in the memory to control the HVAC; determining the setpoints for the HVAC using the wireless sensors placed indoors and algorithms on the smart phone;

8. The method of claim 7 wherein the CCU uses the wireless technology based on 802.11 b/g/n.

9. The method of claim 7 wherein the CCU is powered using the power from the control wires of the HVAC.

10. The method of claim 7 wherein the CCU does not have any display or user interface.

11. The method of claim 7 wherein the CCU is secured using one of the commercially available WiFi encryptions.

12. The method of claim 7 wherein the CCU is the hub for all the wireless sensors used for determining the environment conditions.

13. The method of claim 7 wherein the smart device is used as the storage, processing and computational hub for determining the setpoints.

14. The method of claim 7 wherein the setpoints are recommended using the smart device that optimizes between comfort and energy efficiency.

15. The method of claim 7 wherein the remote monitoring is accomplished using a server that is used as a storage hub for sensor data and accessible by the smart device.
